(19) United States
(12) Patent Application Publication
(10) Pub. No.: US 2005/0276849 A1
(43) Pub. Date: Dec. 15, 2005

(54) Sustained release dosage forms

(76) Inventors: Nilobon Podhipleux, Weston, FL (US); Unchalee Lodin, North Miami Beach, FL (US); Chih-Ming Chen, Taipei (TW); Avinash Nangia, Weston, FL (US)

Correspondence Address: DAVIDSON, DAVIDSON & KAPPEL, LLC, 14th Floor, 485 Seventh Avenue, New York, NY, 10018, US

(21) Appl. No.: 10/868,285

(22) Filed: Jun. 15, 2004

(51) Int. Cl. A61K 9/22; A61K 9/50

(52) U.S. Cl. 424/468

(57) ABSTRACT

The present invention is directed to sustained release neutralized divalproex sodium oral dosage forms, processes for preparing the same, and methods of treatment therewith.

### FIELD OF THE INVENTION

The present invention is related to a sustained release oral dosage form comprising neutralized divalproex sodium and a solubility modulating agent. Preferably the inclusion of the solubility modulating agent in the dosage form provides for a release profile that is therapeutically desirable.

### BACKGROUND OF THE INVENTION

Valproic acid, or 2-propylpentanoic acid, and its salts and derivatives are compounds with anticonvulsant properties. Of these, valproic acid and its sodium salt (sodium valproate) are the most well known. U.S. Pat. No. 3,325,361 describes the use of valproic acid, sodium valproate and other salts and derivatives of valproic acid as anti-convulsants. All documents cited herein, including the foregoing, are incorporated by reference in their entireties for all purposes.

It has been recognized by those skilled in the art that both valproic acid and sodium valproate are difficult to formulate into solid oral dosage forms. Valproic acid, for example, is an oily liquid. Sodium valproate is known to be very hygroscopic and to liquify rapidly, and is, therefore, difficult to formulate into tablets.

Efforts have been made to address the problems associated with formulating valproic acid and sodium valproate into solid oral dosage forms. U.S. Pat. No. 5,049,586 (Ortega, et al.) describes valproic acid tablets having a specific composition, which tablets are said to be stable. The tablets contain valproic acid, magnesium oxide, corn starch, poyvinylpyrrolidone, sodium carboxymethylcellulose, and magnesium stearate in specific proportions.

U.S. Pat. No. 5,017,613 (Aubert, et al.) describes a process for preparing a composition containing valproic acid in combination with valproate sodium, wherein the process does not use any binder or granulating solvent. In the process, a mixture of valproic acid and ethylcellulose is prepared and valproate sodium is added to the mixture to form drug granules in the absence of any binder or granulating solvent. Precipitated silica is added to the granules before the compression into tablets.

Efforts have also been made to overcome the limited utility of valproic acid and sodium valproate in formulating solid dosage forms by creating a different salt form or a derivative of valproic acid. U.S. Pat. No. 4,895,873 (Schafer) describes a crystalline calcium salt of valproic acid, in which five valproic acid radicals are associated with one calcium ion. The crystalline salt, called calcium pentavalproate, is said to be non-hygroscopic.

U.S. Pat. No. 4,558,070 (Bauer, et al.) describes potassium, cesium or rubidium salt of valproic acid, which is prepared by combining 4 moles of valproic acid with 1 mole of the potassium, cesium or rubidium. U.S. Pat. No. 4,699,927 (Deboeck) describes arginine, lysine, histidine, ornithine or glycine salts of valproic acid.

U.S. Pat. Nos. 5,212,326 and 4,988,731 (Meade) describe divalproex sodium and its preparation. Divalproex sodium is described as an ionic oligomer in which one mole each of the valproic acid form coordinate bonds with the sodium of the sodium valproate molecule, where the valproate ion is ionically bonded to the sodium ion. Meade also describes the oligomeric compound as having better physical properties than either monomer from which it is made in that the oligomer is a crystalline, non-hygroscopic, stable solid compound.

Some patents describe sustained release dosage forms for divalproex sodium, valproic acid, its salts, amides, or other derivatives. U.S. Pat. No. 5,980,943 (Ayer, et al.) describes a sustained release delivery device for administering divalproex sodium, valproic acid, and its salts and derivatives. The device comprises a semipermeable wall containing drug granules that are microencapsulated with polyalkylene oxide or carboxymethylcellulose polymer.

U.S. Pat. No. 4,913,906 (Friedman, et al.) describes a controlled release dosage form containing divalproex sodium, valproic acid, valpromide and other valproic acid salts and derivatives. The composition is prepared by mixing the drug with hydroxypropyl cellulose, ethylcellulose, or esters of acrylic and methacrylic acid, and by applying high pressure to the mixture of the ingredients.

U.S. Pat. No. 5,807,574 (Cheskin, et al.) describes a controlled release dosage form containing divalproex sodium and a process for its preparation. The process involves melting divalproex sodium and mixing it with a molten wax to form a divalproex sodium-wax composite. The drug-wax mixture is formulated into a capsule.

U.S. Pat. No. 5,169,642 (Brinker, et al.) describes a sustained release dosage form containing granules of divalproex sodium, valproic acid or amides or esters or salts thereof and a polymeric viscosity agent. The drug is coated with a sustained release composition comprising specified portions of ethylcellulose or a methacrylic methylester, plasticizer, and detactifying agent.

U.S. Pat. No. 5,068,110 (Fawzi, et al.) describes various delayed-release tablets and capsules currently marketed, including the delayed-release divalproex sodium tablets manufactured by Abbott Laboratories, and states that the stability of an enteric coated capsules is increased by the application of thicker, higher levels of the enteric coating having a thickness of 14 mg/cm² to 24 mg/cm², alone or in combination with a hydroxypropylcellulose, hydroxymethylcellulose or hydroxypropylmethyl cellulose coating.

There exists a need in the art to formulate a sustained release oral dosage form comprising a neutralized form of divalproex sodium that provides a therapeutically desirable release profile.

### OBJECTS AND SUMMARY OF THE INVENTION

It is an object of the invention to provide a sustained release oral dosage form comprising neutralized divalproex sodium and a solubility-modulating agent.

It is an object of certain embodiments of the present invention to provide a process for preparing a sustained release oral dosage form comprising neutralized divalproex sodium and a solubility modulating agent.

It is an object of certain embodiments of the present invention to provide a sustained release oral dosage form comprising a therapeutically effective amount of divalproex sodium; and a solubility-modulating agent; wherein the divalproex sodium is not present as an oligomeric structure or a 1:1 molar ratio of sodium valproate to valproic acid.

It is an object of certain embodiments of the present invention to provide a dosage form for controlled delivery of neutralized divalproex sodium where the delivery profile of said neutralized divalproex sodium is controlled by the dosage form and not by the intrinsic water solubility of the drug.

It is an object of certain embodiments of the present invention to provide a method for converting an unacceptable neutralized divalproex sodium release profile into a profile that is recognized as therapeutically desirable. For example, neutralized divalproex sodium having an intrinsic water solubility that is very high will release from an osmotic oral dosage form at a high rate; modulation to decrease the solubility of neutralized divalproex sodium will decrease the release rate into the therapeutic range over a sustained period of time. Preferably, the modulation of the neutralized divalproex sodium is achieved without chemical modification of the neutralized divalproex sodium.

The above objects and others are attained by virtue of the present invention which is directed in part to a sustained release oral dosage form comprising neutralized divalproex sodium and a solubility modulating agent, said dosage form providing for the sustained release of the neutralized divalproex sodium over a period of about 8 to about 24 hours or more.

In certain embodiments, the sustained release oral dosage form comprising the neutralized divalproex sodium and the solubility modulating agent is overcoated with a semipermeable membrane.

In preferred embodiments, the sustained release oral dosage form of the present invention comprises:
a core comprising (i) a therapeutically effective amount of neutralized divalproex sodium; and (ii) a solubility modulating agent;
a membrane coating surrounding the core, said membrane coating having at least one passageway; and
the dosage form providing a time to maximum plasma concentration (Tₘₐₓ) of the drug at from about 4 to about 20 hours after administration to a human patient.

In certain embodiments, the solubility of the sustained release oral dosage form comprising the neutralized divalproex sodium is slowed by inclusion of the solubility modulating agent in the dosage form.

In certain embodiments, the present invention is further directed to a process for preparing a neutralized divalproex sodium sustained release oral dosage form of the present invention.

Preferably the process of the present invention comprises preparing a neutralized divalproex sodium solution or dispersion by combining divalproex sodium, having a sodium valproate moiety and a valproic acid moiety, with a base and optionally an aqueous solvent. The base (e.g., sodium hydroxide) is added in a sufficient amount to ensure neutralization of the valproic acid moiety of the divalproex sodium. In the neutralized divalproex sodium solution, divalproex sodium does not retain its oligomeric structure and does not have a 1:1 molar ratio of sodium valproate and valproic acid. The process further comprises granulating the neutralized divalproex sodium solution or dispersion with a pharmaceutically acceptable carrier and mixing the granulation with a solubility modulating agent and forming a sustained release oral dosage form.

In certain preferred embodiments, the solubility modulating agent of the present invention is coated with a water insoluble coating prior to mixing the solubility modulating agent with the granules comprising the neutralized divalproex sodium.

In certain embodiments, additional processing steps are undertaken to prepare a uniform granulate suitable for formulating into tablets. For example, sufficient quantities of pharmaceutically acceptable tableting excipients may be admixed with the neutralized divalproex sodium and solubility modulating agent granulation, and the resulting mixture is compressed into tablets.

In certain embodiments of the invention, the dosage form provides an in-vitro release rate, of neutralized divalproex sodium, when measured by the USP Basket Method at 100 rpm in 900 ml at a pH 7.5 phosphate buffer at 37° C. of from 10% to about 50% at 2 hours, from about 10% to about 60% at 4 hours, from about 20% to about 80% at 8 hours, from about 30% to about 90% at 12 hours, from about 40% to about 90% at 20 hours, and greater than about 50% at 24 hours.

In certain preferred embodiments of the invention, the dosage form provides an in-vitro release rate, of neutralized divalproex sodium, when measured by the USP Basket Method at 100 rpm in 900 ml at a pH 7.5 phosphate buffer at 37° C. of from about 10% to about 40% at 2 hours, from about 20% to about 60% at 4 hours, from about 30% to about 70% at 8 hours, from about 40% to about 80% at 12 hours, from about 50% to about 90% at 20 hours, and greater than about 50% at 24 hours.

In certain embodiments, the invention is directed to a method of treating a human patient, comprising orally administering a sustained release oral dosage form as described herein to a patient in need of such treatment.

In certain embodiments, the present invention is further directed to a method of treating and/or preventing complex partial seizures, mania associated with bipolar disorders, and/or migraine headaches in a human patient comprising orally administering a sustained release oral solid dosage form as described herein.

The term “neutralized divalproex sodium,” for purposes of the present invention, refers to divalproex sodium in which the valproic acid moiety has been neutralized by the addition of a base, e.g., sodium hydroxide. Neutralized divalproex sodium is not an oligomer. Neutralized divalproex sodium also does not exhibit a 1:1 molar ratio of sodium valproate and valproic acid. Sustained release neutralized divalproex sodium oral dosage forms prepared using neutralized divalproex sodium solution, therefore, do not contain oligomeric divalproex sodium, nor do they exhibit 1:1 molar ratio of sodium valproate and valproic acid.

The term “sustained release” for purposes of the present invention means that the therapeutically active medicament (i.e., neutralized divalproex sodium) is released from the formulation at a controlled rate such that therapeutically beneficial blood levels (but below toxic levels) of the medicament are maintained over an extended period of time, e.g., providing a 24 hour therapeutic effect.

The term “environmental fluid” for purposes of the present invention is meant to encompass, e.g., an aqueous solution, such as that used for in-vitro dissolution testing, or gastrointestinal fluid.

The term “Cₘₐₓ” is meant for purposes of the present invention to mean then maximum plasma concentration of a medicament achieved after administration of a dosage form in accordance with the present invention.

The term “Tₘₐₓ” is meant for purposes of the present invention to mean the elapsed time from administration of a dosage form to the time the Cₘₐₓ of the medicament is achieved.

The term “mean” for purposes of the present invention, when used to define a pharmacokinetic value (e.g., Tₘₐₓ) represents the arithmetic mean value measured across a patient population.

### BRIEF DESCRIPTION OF THE DRAWINGS

FIG. 1 is a graphical representation of the dissolution profile of neutralized divalproex sodium tablets of Examples 1, 2 and 3 in pH 7.5 phosphate buffer as compared to reference standard Depakote® ER (basket method at 75 rpm).

### DETAILED DESCRIPTION OF THE INVENTION

Divalproex sodium is a oligomer having a 1:1 molar ratio of sodium valproate and valproic acid. The oligomer is described as a stable crystalline solid and is designated as sodium hydrogen bis (2-propyl pentanoate).

Upon administration, divalproex dissociates into valproate ion in the gastrointestinal tract, and in that form exerts its pharmacological effect. Divalproex sodium is indicated for the treatment of patients with complex partial seizures, as well as for the treatment of mania associated with bipolar disorders and for prophylaxis of migraine headaches.

U.S. Pat. No. 4,558,070 (Bauer, et al.) indicates that divalproex sodium is a highly stable, non-hygroscopic, crystalline compound. Bauer also discusses a theory behind the stability of divalproex sodium, stating that it is not a mixture of the two precursors but a chemical entity, and that in the oligomer, the outer shell of electrons of the sodium atom is filled by coordination to the oxygen atoms of both valproic acid and valproate ions, resulting in a stable complex where the sodium ion is completely surrounded by oxygen. Bauer, et al., therefore, appears to indicate that the particular oligomeric structure and the molar ratio of divalproex sodium accounts for the stability of the compound.

The sustained release oral dosage forms of the present invention comprises neutralized divalproex sodium and a solubility modulating agent. Preferably the sustained release oral dosage form of the present invention comprises (i) a core tablet comprising neutralized divalproex sodium and a solubility modulating agent; and (ii) a membrane wall coated over said core tablet.

Neutralized divalproex sodium of the present invention, is preferably in the form of a neutralized divalproex sodium solution prepared by combining divalproex sodium with a base and an aqueous solvent. The base is added in sufficient quantities to ensure neutralization of the valproic acid moiety of the divalproex sodium. In certain preferred embodiments, the pH of the neutralized divalproex sodium solution is about 10.8±1.0, most preferably 10.8±0.5.

The base used in the present invention in the dissolution and neutralization of the divalproex sodium can be any pharmaceutically acceptable base such as sodium carbonate, sodium bicarbonate, sodium phosphate dibasic, sodium phosphate tribasic, sodium citrate, magnesium hydroxide, magnesium carbonate, calcium carbonate, calcium phosphate, sodium hydroxide, mixtures thereof, and the like. A most preferred base is sodium hydroxide.

Preferably, the basic solution comprises sodium hydroxide as a base and water as an aqueous solvent, although other aqueous solvents may be used. In certain embodiments, additional sodium hydroxide or additional water may be added to the neutralized divalproex sodium solution preferably providing a resulting solution that has 20-60%, most preferably 50±3%, valproic acid activity.

In accordance with certain preferred embodiments of the present invention, the neutralized divalproex sodium solution is granulated with a pharmaceutically acceptable carrier. Granulation techniques are well known in the art and include for example, wet granulation, spray granulation and the like. Preferably, the solution of the neutralized divalproex sodium is spray granulated with the carrier and dried to produce divalproex sodium granules. The granules may then be sized through an appropriate sized screen, e.g., a 16 mesh screen. Alternatively, a spray coating system can be used to produce divalproex sodium coated substrates, utilizing, e.g., inert beads as the substrates.

Examples of pharmaceutically acceptable carriers for use in the present invention include, but are not limited to, calcium phosphate dihydrate, calcium sulfate dihydrate, microcrystalline cellulose, cellulose derivatives, dextrose, lactose, anhydrous lactose, spray-dried lactose, lactose monohydrate, mannitol, starches, sorbitol and sucrose, hydroxypropylmethylcellulose, hydroxypropylcellulose, methyl cellulose, carboxymethyl cellulose, sodium carboxymethyl cellulose, polyvinylpyrrolidone, polyethyleneglycol, cellulose acetate butyrate, hydroxyethyl cellulose, ethyl cellulose, polyvinyl alcohol, polypropylene, dextrans, dextrins, hydroxypropyl-beta-cyclodextrin, chitosan, copolymers of lactic and glycolic acid, lactic acid polymers, glycolic acid polymers, polyorthoesters, polyanyhydrides, polyvinyl chloride, polyvinyl acetate, ethylene vinyl acetate, lectins, carbopols, silicon elastomers, polyacrylic polymers, maltodextrins, fructose, inositol, trehalose, maltose raffinose, and alpha-, beta-, and gamma-cyclodextrins, and suitable mixtures of the foregoing. A preferred pharmaceutically acceptable carrier is anhydrous lactose, or dextrose.

Preferably, the pharmaceutically acceptable carrier comprises a plurality of particles, and the divalproex sodium solution is sprayed onto the carrier and dried to produce divalproex sodium granules. Thereafter, the solubility modulating agent is mixed with the divalproex sodium granules.

Examples of solubility modulating agents for use in the present invention are organic carboxylic acids such as, for example and without limitation, citric acid, tartaric acid, malic acid, fumaric acid, adipic acid, succinic acid, and ascorbic acid; acid anhydrides such as succinic anhydride and citric anhydride, and acid salts such as sodium dihydrogen phosphate, disodium dihydrogen pyrophosphate, sodium acid suphite, monopotassium citrate, potassium acid tartrate and sodium fumarate; suitable carbonate sources such as sodium bicarbonate, sodium carbonate, potassium bicarbonate, potassium carbonate, sodium sesquicarbonate, sodium glycine carbonate, calcium bicarbonate, calcium carbonate and magnesium carbonate; mixtures thereof and the like.

Preferably the solubility modulating agent is surrounded by a water insoluble coat such as acrylic resins such as Eudragit L®, shellac, cellulose acetate butyrate, hydroxypropyl methylcellulose phthalate, cellulose acetyl phthalate, cellulose triacetyl phthalate, sodium cellulose acetate phthalate, cellulose ester phthalate, cellulose ether phthalate, methylcellulose phthalate, cellulose ester-ether phthalate, hydroxy propyl cellulose phthalate, alkali salts of cellulose acetate phthalate, alkaline earth salts of cellulose acetate phthalate, calcium salt of cellulose acetate phthalate, ammonium salt of hydroxypropyl methylcellulose phthalate, cellulose acetate hexahydrophthalate, hydroxypropyl methylcellulose hexahydrophthalate, polyvinyl acetate phthalate, mixtures thereof, and the like.

The coating is preferably applied prior to mixing the solubility modulating agent with the granules comprising the neutralized divalproex sodium. The coating may be applied to the solubility modulating agent by press coating, molding, spraying, dipping and/or air-suspension or air tumbling procedures or other procedures known in the art. A preferred method of applying the coating is by fluidized bed coating, where the coating is applied by spraying the coating composition onto the solubility modulating agent using a Wurster apparatus. The coating may be applied to the solubility modulating agent by employing solvents, including an organic, aqueous or a mixture of an organic and aqueous solvent. Examplary solvents suitable in applying the coating include an alcohol, ketone, ester, ether, aliphatic hydrocarbon, halogenated solvents, cycloaliphatic solvents, aromatic, heterocyclic, aqueous solvents, mixtures thereof, and the like.

In certain embodiments, a portion of the solubility modulating agent may be left uncoated to affect immediate availability during the period intervening the onset of release from the sustained release solubility modulating element(s) upon exposure to an environmental fluid. In certain embodiments, the solubility modulating agent can also be incorporated into an individual matrix; preferably the incorporation into the individual matrix affects the release of the solubility modulating agent.

Materials suitable as matrix materials for dispersing the solubility modulating agents include those described previously for use as coating materials. Additional appropriate matrix materials include materials that are semisolid to solid that dissolve or erode within the fluid which forms within the core in the environment of use, or insoluble materials that serve as diffusion media to modulate the leaching of the solubility modulator into the core compartment fluids. Specific examples include, but are not limited to hydroxypropyl cellulose, hydroxypropylmethyl cellulose, solid polyethylene glycols, carboxypolymethylene, silicone rubber, pectin ethylene vinyl acetate, waxes, fats, fatty acids, fatty alcohols, triglycerides, natural gums, polylactic acid, poly(ortho ester)s, and the like.

The final dosage form may contain solubility modulators in the various form of either: (a) coated solubility modulator; (b) solubility modulator dispersed in a matrix; (c) immediate availability solubility modulator; or (d) mixtures thereof.

In certain embodiments, the dosage form further comprises an osmagent. Any osmagent known in the art may be used. As stated above, the solubility modulating agent may also act as the osmagent or there may be a separate osmagent. In certain embodiments, the neutralized divalpreox sodium may also act as the osmagent, by itself, or in combination with the solubility modulating agent. For example, the solubility modulating agent may alter the solubility of the neutralized divalproex sodium causing it to act as the osmagent. The osmagent is a substance which, in solution, exhibits a certain osmotic pressure that is the driving force for water ingress into the sustained release oral dosage form (this increases the internal hydrostatic pressure resulting in release of a substance through a barrier membrane). Exemplary osmotic agents include, for example and without limitation, sugars such as sucrose, lactose, mannitol, maltose, sorbitol and fructose; neutral salts such as sodium chloride, magnesium sulfate, magnesium chloride, potassium sulfate, sodium carbonate, sodium sulfite, potassium acid phosphate, sodium acetate and ethyl acetate; acidic components such as fumaric acid, maleic acid, adipic acid, citric acid and ascorbic acid; alkaline components such as tris(hydroxylmethyl) aminomethane (TRIS); meglumine, tribasic and dibasic phosphates of sodium and potassium; amino acids such as glycine and arginine; and other compounds such as urea; mixtures thereof, and the like.

In certain embodiments other excipients can be combined with the neutralized divalproex sodium and the solubility modulating agent as needed for example to control pH, promote stability, facilitate manufacturability, and/or provide osmotic activity to the dissolved core compartment solution to effect a desirable release profile. The entire composite may then be compressed or formed into core tablets onto which a membrane wall containing optional leachable pore forming additives is applied.

In certain preferred embodiments, a membrane wall is applied to the core tablet. The membrane wall preferably comprises a polymer permeable to water but impermeable to solute. Examples of such polymers include, for example and without limitation, cellulose acetate having a degree of substitution, D.S., meaning the average number of hydroxyl groups on the anhydroglucose unit of the polymer replaced by a substituting group, up to 1 and acetyl content up to 21%; cellulose diacetate having a D.S. of 1 to 2 and an acetyl content of 21 to 35%; cellulose triacetate having a D.S. of 2 to 3 and an acetyl content of 35 and 44.8%; cellulose propionate having an acetyl content of 1.5 to 7% and a propionyl content of 2.5 to 3% and an average combined propionyl content of 39.2 to 45% and a hydroxyl content of 2.8 to 5.4%; cellulose acetate butyrate having a D.S. of 1.8, an acetyl content of 13 to 15%, and a butyryl content of 34 to 39%; cellulose acetate having an acetyl content of 2 to 99.5%, a butyryl content of 17 to 53%, and a hydroxyl content of 0.5 to 4.7%; cellulose triacylates having a D.S. of 2.9 to 3 such as cellulose trivalerate, cellulose trilaurate, cellulose tripalmitate, cellulose trisuccinate, cellulose triheptylate, cellulose tricaprylate, cellulose trioctanoate, and cellulose tripropionate; cellulose diesters having a lower degree of substitution and prepared by the hydrolysis of the corresponding triester to yield cellulose diacylates having a D.S. of 2.2 to 2.6 such as cellulose dicaprylate and cellulose dipentanate; and esters prepared from acyl anhydrides or acyl acids in an esterification reaction to yield esters containing different acyl groups attached to the same cellulose polymer such as cellulose acetate valerate, cellulose acetate succinate, cellulose propionate succinate, cellulose acetate octanoate, cellulose valerate palmitate, cellulose acetate palmitate and cellulose acetate heptanoate; mixtures thereof; and the like. The most preferred polymer material is cellulose acetate comprising an acetyl content of 39.3 to 40.3%, commercially available from Eastman Fine Chemicals.

Additional polymers that can be used for the purpose of the invention include cellulose acetate acetoacetate, cellulose acetate chloroacetate, cellulose acetate furoate, dimethoxyethyl cellulose acetate, cellulose acetate carboxymethoxypropionate, cellulose acetate benzoate, cellulose butyrate naphthylate, cellulose acetate benzoate, methylcellulose acetate, methylcyanoethyl cellulose, cellulose acetate methoxyacetate, cellulose acetate ethoxyacetate, cellulose acetate dimethylsulfamate, ethylcellulose, ethylcellulose dimethylsulfamate, cellulose acetate p-toluene sulfonate, cellulose acetate methylsulfonate, cellulose acetate dipropylsulfamate, cellulose acetate butylsulfonate, cellulose acetate laurate, cellulose stearate, cellulose acetate methylcarbamate, agar acetate, amylose triacetate, beta glucan acetate, beta glucan triacetate, acetaldehyde dimethyl acetate, cellulose acetate ethyl carbamate, cellulose acetate phthalate, cellulose acetate dimethyl aminoacetate, cellulose acetate ethyl carbonate, poly (vinyl methyl) ether copolymers, cellulose acetate with acetylated hydroxyethyl cellulose, hydroxylated ethylenevinyl acetate, poly(ortho ester)s, polyacetals, semipermeable polyglycolic or polylactic acid and derivatives thereof, film forming materials with a water sorption of one to fifty percent by weight at ambient temperatures with a presently preferred water sorption of less than thirty percent, acylated polysaccharides, acylated starches, aromatic nitrogen containing polymeric materials that exhibit permeability to aqueous fluids, membranes made from polymeric epoxides, copolymers of alkylene oxides and alkyl glycidyl ethers, polyurethanes, polyacrylate and polymethacrylate polymers, mixtures thereof, derivatives thereof, and the like. Admixtures of the various polymers described herein may also be used.

The polymers described are known to the art or they can be prepared according to the procedures in Encyclopedia of Polymer Science and Technology, Vol. 3, pages 325 to 354, and 459 to 549, published by Interscience Publishers, Inc., New York, in Handbook of Common Polymers by Scott, J. R. and Roff, W. J., 1971, published by CRC Press, Cleveland, Ohio; and in U.S. Pat. Nos. 3,133,132; 3,173,876; 3,276,586; 3,541,055; 3,541,006; and 3,546,142.

In addition, the membrane can be formed with enteric material. As enteric coating material polymers one or more, separately or in combination, of the following can be used; e.g. solutions or dispersions of methacrylic acid copolymers, cellulose acetate phthalate, hydroxypropyl methylcellulose phthalate, hydroxypropyl methylcellulose acetate succinate, polyvinyl acetate phthalate, cellulose acetate trimellitate, carboxymethylethylcellulose, shellac or other suitable enteric coating layer polymer(s). Some preferred commercial enteric coating materials are EUDRAGIT® L 100-55, EUDRAGIT® L 30 D-55, EUDRAGIT® L 100, and EUDRAGIT® S 100.

The enteric coating material comprises approximately 0 to about 60% of the total weight of the coating, most preferably about 2% to about 40% of the total weight of the coating.

In certain preferred embodiments of the present invention, the oral dosage form contains at least one passageway in the membrane. As used herein the term passageway includes an aperture, orifice, bore, hole, weaken area or an erodible element such as a gelatin plug that erodes to form an osmotic passageway for the release of the neutralized divalproex sodium. A detailed description of the passageway can be found in U.S. Pat. Nos. such as 3,845,770, 3,916,899, 4,034,758, 4,063,064, 4,077,407, 4,088,864, 4,783,337 and 5,071,607 (the disclosures of which are hereby incorporated by reference).

In certain embodiments the passageway can be formed by drilling, including mechanical and laser drilling, through the membrane. Passageways and equipment for forming passageways are disclosed in U.S. Pat. Nos. 3,845,770, 3,916,899, 4,063,064, and 4,088,864. In certain preferred embodiments, a passageway is drilled in each side of the tablet.

In other embodiments, the passageway is formed by making an indentation onto the core prior to the membrane coating to form a weakened area of the membrane at the point of the indentation.

The membrane wall of the dosage form may additionally comprise a plasticizer. Exemplary plasticizers suitable for the present purpose include plasticizers that lower the temperature of the second-order phase transition of the wall or the elastic modulus thereof, and also increase the workability of the wall and its flexibility. Plasticizers may increase or decrease the permeability of the wall to fluids including water and aqueous solutions. Plasticizers operable for the present purpose include both cyclic plasticizers and acyclic plasticizers. Typical plasticizers are those selected from the group consisting of phthalates, phosphates, citrates, adipates, tartrates, sebacates, succinates, glycolates, glycerolates, benzoates, myristates, polyethylene glycols, polypropylene glycols, and halogenated phenyls, and the like. Certain preferred plasticizers are triacetin, acetylated monoglyceride, grape seed oil, olive oil, sesame oil, acetyltributylcitrate, acetyltriethylcitrate, glycerin sorbitol, diethyloxalate, diethylmalate, diethylfumarate, dibutylsuccinate, diethylmalonate, dioctylphthalate, dibutylsebacate, triethylcitrate, tributylcitrate, glyceroltributyrate, mixtures thereof, and the like.

Depending on the particular plasticizer, amounts of from 0 to about 50%, and preferably about 2% to about 30% of the plasticizer can be used based upon the total weight of the coating.

Suitable plasticizers can be selected for blending with the membrane wall materials by selecting plasticizers that have a high degree of solvent power for the materials, are compatible with the materials over both the processing and use temperature range, exhibit permanence as seen by their tendency to remain in the plasticized wall, impart flexibility to the material and are non-toxic to humans.

In certain embodiments, the membrane further comprises a flux enhancing agent. The flux enhancing agent increases the volume of fluid imbibed into the core to enable the dosage form to dispense substantially all of the neutralized divalproex sodium through the passageway and/or the porous membrane. The flux enhancing agent can be a water soluble material or an enteric material. Some examples of the preferred materials that are useful as flux enhancers are sodium chloride, potassium chloride, sucrose, sorbitol, mannitol, polyethylene glycol (PEG), propylene glycol, hydroxypropyl cellulose, hydroxypropyl methycellulose, hydroxyprophy methycellulose phthalate, cellulose acetate phthalate, polyvinyl alcohols, methacrylic acid copolymers and mixtures thereof. A preferred flux enhancer is PEG 400.

The flux enhancer may also be a drug that is water soluble or a drug that is soluble under intestinal conditions. If the flux enhancer is a drug, the present dosage form has the added advantage of providing an immediate release of a drug which is selected as the flux enhancer.

In certain embodiments, the flux enhancing agent comprises approximately 0 to about 40% of the total weight of the coating, most preferably about 2% to about 20% of the total weight of the coating. The flux enhancing agent preferably dissolves or leaches from the membrane to form paths in the membrane for the fluid to enter the core and dissolve the active ingredient.

The membrane may be further coated with a pharmaceutically acceptable film-coating, e.g., for stability purposes (e.g., coated with a moisture barrier), etc. For example, the membrane may be overcoated with a film coating, preferably containing a pigment and a barrier agent, such as hydroxypropylmethylcellulose and/or a polymethylmethacrylate. An example of a suitable material which may be used for such a hydrophilic coating is hydroxypropylmethylcellulose (e.g., Opadry®, commercially available from Colorcon, West Point, Pa.). In addition, other suitable materials are Povidone K30, PEG 3350, or the like. Any pharmaceutically acceptable manner known to those skilled in the art may be used to apply the coatings. For example, the coating may be applied using a coating pan or a fluidized bed. An organic, aqueous or a mixture of an organic and aqueous solvent is used for the hydrophobic polymer or enteric coating. Examples of suitable organic solvents are, e.g., isopropyl alcohol, ethanol, and the like, with or without water. Aqueous solvents are preferred for the overcoating procedures. In certain preferred embodiments, the pharmaceutically acceptable film-coating around the membrane does not affect, or does not substantially affect the release of the active agent from the dosage form.

In certain embodiments, the membrane coating around the core is less than 10% of the total weight of the dosage form, preferably the membrane coating around the core will be from about 1% to about 7%, preferably from about 1% to about 5%, most preferably from about 1% to about 4% based on the total weight of the formulation.

In certain embodiments, the membrane is permeable to aqueous fluids or gastrointestinal fluids, but not to the active agent. Thus, in certain embodiments, the drug is release through the at least one hole or passageway in the membrane.

In certain alternate embodiments, the membrane is permeable to both aqueous solutions or gastrointestinal fluids and to the active agent. Thus, the membrane is permeable to the active agent and drug is released through a hole or passageway and through the membrane in the environmental fluid.

In certain embodiments, the dosage form of the present invention may also comprise an effective amount of the drug that is available for immediate release. The effective amount of drug available for immediate release may be coated onto the membrane or the dosage form or it may be incorporated into the membrane.

In certain embodiments, optional pharmaceutical excipients are added to the mixture of neutralized divalproex sodium granules and solubility modulating agent in the process of formulating the mixture into tablet or tablet cores. Such pharmaceutical excipients may include but are not limited to a lubricant, disintegrant, binder, glidant and/or diluent.

Examples of lubricants include magnesium stearate, calcium stearate, oleic acid, caprylic acid, stearic acid, magnesium isovalerate, calcium laurate, magnesium palmitate, behenic acid, glyceryl behenate, glyceryl stearate, sodium stearyl fumarate, potassium stearyl fumarate, and zinc stearate.

Suitable disintegrants include crospovidone, alginates, cellulose and its derivatives, clays, polyvinylpyrrolidone, polysaccharides, such as corn and potato starch, dextrins and sugars. Disintegrants, when used in the formulation, facilitates disintegration when the tablet contacts water in the gastrointestinal tract.

Binders, when added to the formulation, promote granulation and/or promote cohesive compact during the direct compression into tablets. Examples of binders include acacia, cellulose derivatives, gelatin, glucose, polyvinylpyrrolidone, sodium alginate and alginate derivatives, sorbitol, starch, mixtures thereof, and the like.

Examples of glidants include but are not limited to corn starch, silica derivatives (e.g., colloidal silicon dioxide), talc, and the like.

Examples of inert diluents can include the pharmaceutically acceptable carriers as described above, for example and without limitation, calcium phosphate dihydrate, calcium sulfate dihydrate, microcrystalline cellulose, cellulose derivatives, dextrose, lactose, anhydrous lactose, spray-dried lactose, lactose monohydrate, mannitol, starches, sorbitol and sucrose. Further examples of the carrier include hydroxypropylmethylcellulose, hydroxypropylcellulose, methyl cellulose, carboxymethyl cellulose, sodium carboxymethyl cellulose, polyvinylpyrrolidone, polyethyleneglycol, cellulose acetate butyrate, hydroxyethyl cellulose, ethyl cellulose, polyvinyl alcohol, polypropylene, dextrans, dextrins, hydroxypropyl-beta-cyclodextrin, chitosan, copolymers of lactic and glycolic acid, lactic acid polymers, glycolic acid polymers, polyorthoesters, polyanyhydrides, polyvinyl chloride, polyvinyl acetate, ethylene vinyl acetate, lectins, carbopols, silicon elastomers, polyacrylic polymers, maltodextrins, fructose, inositol, trehalose, maltose raffinose, and alpha-, beta-, and gamma-cyclodextrins, suitable mixtures of the foregoing, and the like.

In accordance with the invention, the neutralized divalproex sodium tablet cores may further be coated with a seal coating. In a preferred embodiment, the seal coating occurs between the tablet core and the membrane coating. The seal coating may comprise a hydrophilic polymer, such as for example and without limitation, hydroxypropyl cellulose, hydroxypropylmethylcellulose, methoxypropyl cellulose, hydroxypropylisopropylcellulose, hydroxypropylpentylcellulose, hydroxypropylhexylcellulose mixtures thereof, and the like. The seal coating may be mixed with other suitable excipients described above (e.g., binders, lubricants, etc.), prior to application.

The seal coating may be applied by press coating, molding, spraying, dipping and/or air-suspension or air tumbling procedures. In a preferred embodiment, the seal coating comprises hydroxypropyl cellulose and hydroxypropylmethylcellulose, and is delivered as a suspension using ethanol as a solvent.

The sustained release neutralized divalproex sodium tablets may be overcoated with a pharmaceutically acceptable film coating, e.g., for aesthetic purposes (e.g., including a colorant), for stability purposes (e.g., coated with a moisture barrier), for taste-masking purposes, etc. For example, the tablets may be overcoated with a film coating, preferably containing a pigment and a barrier agent, such as hydroxypropylmethycellulose and/or a polymethylmethacrylate. An example of a suitable material which may be used for such overcoating is hydroxypropylmethylcellulose (e.g., Opadry®, commercially available from Colorcon, West Point, Pa.). In a preferred embodiment, an overcoating is applied to the divalproex sodium tablets that have already been coated with a seal coating and an enteric coating. The overcoat may be applied using a coating pan or a fluidized bed, and may be applied by using a solvent, preferably an aqueous solvent.

The final product is optionally subjected to a polishing step to improve the appearance of the final product and also to facilitate the manipulation of the formulation post manufacture. For example, the slippery nature of the polished dosage form aids in filling printer carrier bars with the formulation and facilitates final packaging of the product. Suitable polishing agents are polyethylene glycols of differing molecular weight or mixtures thereof, talc, surfactants (e.g., Brij types, Myrj types, glycerol mono-stearate and poloxamers), fatty alcohols (e.g., stearyl alcohol, cetyl alcohol, lauryl alcohol and myristyl alcohol) and waxes (e.g., carnauba wax, candelilla wax and white wax). Preferably, polyethylene glycols having molecular weight of 3,000-20,000 are employed.

In certain preferred embodiments, the present invention provides a process for preparing neutralized divalproex sodium sustained release tablets. The process comprises preparing a neutralized divalproex sodium solution by combining divalproex sodium, having a sodium valproate moiety and a valproic acid moiety, with an aqueous solvent and a base (e.g., sodium hydroxide). The base is added in sufficient amount to ensure neutralization of the valproic acid moiety of the divalproex sodium. The process further comprises granulating the neutralized divalproex sodium solution with a pharmaceutically acceptable carrier, processing the resultant granules with a solubility modulating agent to obtain tablets or tablet cores. Preferably, overcoating the tablet or tablet cores with a membrane material. Forming a passageway in the membrane material for the passage of the neutralized divalproex sodium upon exposure to an environmental fluid.

The sustained release tablet produced does not contain divalproex sodium that is an oligomeric compound and does not have a 1:1 molar ratio of sodium valproate and valproic acid. Rather, the sustained release tablets of the present invention contain divalproex sodium in which the valproic acid moiety has been neutralized.

In certain preferred embodiments, the processing of the neutralized divalproex sodium granules to obtain tablets or tablet cores comprises drying and then screening the divalproex sodium (neutralized) granules, and admixing the screened divalproex sodium granules with a solubility modulating agent, and thereafter adding pharmaceutically necessary excipients and compressing the resulting mixture into tablets. The pharmaceutically acceptable excipients are selected from the group consisting of a lubricant, a disintegrant, a binder, a glidant, an inert diluent, mixtures thereof, and the like. Examples of suitable excipients are listed above.

In certain preferred embodiments, the neutralized divalproex sodium solution is diluted with isopropyl alcohol before it is granulated with the pharmaceutically acceptablet carrier. The granulation is preferably performed by spraying the neutralized divalproex sodium solution onto the pharmaceutically acceptable carrier (e.g., anhydrous lactose) in a fluid bed processor. Alternatively, a spray coating system can be used to produce divalproex sodium coated substrates. The granules or coated substrates can be blended with suitable excipients, e.g., microcrystalline cellulose, magnesium stearate, etc. and compressed into tablet cores. The tablet cores are coated with a seal coating in a coating pan with a solution or dispersion comprising e.g., hydroxypropylmethylcellulose and magnesium stearate in ethanol. A membrane coating, preferably comprising cellulose acetate is then applied, also in a coating pan, and a passageway is formed in the membrane coating e.g., via a laser drill, to allow for the release of the neutralized divalproex sodium upon exposure to an environmental fluid.

### DETAILED DESCRIPTION OF THE PREFERRED EMBODIMENTS

The following examples illustrate various aspects of the present invention. They are not to be construed to limit the claims in any manner whatsoever.

### EXAMPLE 1

Sodium Valproate XT, 576 mg tablet formulations without a solubility modulating agent were prepared and are listed in Table 1.

TABLE 1

| Ingredient | mg/tablet | % w/w |
|---|---|---|
| Sodium Valproate Granules | | |
| Sodium Valproate, EP | 576.11 | 69.91 |
| Lactose Anhydrous, USP | 134.68 | 16.34 |
| Hydroxypropyl Cellulose, NF (Klucel EF) | 37.41 | 4.54 |
| Ethanol-SDA 3A 190 Proof | * | * |
| Sub-total: | 748.20 | 90.79 |
| Sodium Valproate Tablets, 576 mg (Uncoated) | | |
| Sodium Valproate Granules | 748.02 | 90.79 |
| Colloidal Silicon Dioxide, NF (Cab-O-Sil M5) | 11.51 | 1.40 |
| Magnesium Stearate, NF/FCC | 7.67 | 0.93 |
| Sub-total: | 767.20 | 93.12 |
| Sodium Valproate Tablets, 576 mg (Seal Coated) | | |
| Sodium Valproate Tablets, 576 mg (Uncoated) | 767.20 | 93.12 |
| Hydroxypropyl Methylcellulose, USP (HPMC | 11.86 | 1.44 |
| ES) | | |
| Hydroxypropyl Cellulose, NF (Klucel EF) | 11.86 | 1.44 |
| Ethanol-SDA 3A 190 Proof | * | * |
| Sub-total: | 790.92 | 96.00 |
| Sodium Valproate XT Tablets, 576 mg (CA Coated) | | |
| Sodium Valproate Tablets, 576 mg (Seal Coated) | 790.92 | 96.00 |
| Cellulose Acetate 398-10, NF | 28.01 | 3.40 |
| Triacetin, USP | 1.65 | 0.20 |
| Polyethylene Glycol 400, NF | 3.30 | 0.40 |
| Acetone, NF | * | * |
| Total: | 823.88 | 100.00 |

*Evaporated during processing.
**Used to adjust the batch size to obtain optimum load.
(a) Total batch size for the specified step only. Solvent weight is not included in calculation of batch size as it is evaporated during processing.
(b) Total batch size for the specified step only.

The tablets of having the formulation table 1 are prepared as follows:

1. Wet granulate the requisite amounts of Sodium Valproate, EP; Lactose anhydrous, USP; Hydroxypropyl cellulose, NF (Klucel EF), and ethanol-SDA 3A 190 proof to form sodium valproate granules.

2. Blend the sodium valproate granules with requisite amounts of colloidal silicon dioxide, NF (Cab-O_sil M5) and magnesium stearate, NF/FCC.

3. Compress the blend to form sodium valproate tablets.

4. Coat the sodium valproate tablets with a seal coating comprising requisite amounts of hydroxypropyl methylcellulose, USP(HPMC E5), hydroxypropyl cellulose (Klucel EF) and ethanol-SDA 3A 190 proof to form seal coated tablets.

5. Coat the seal coated tablets with a polymer coating comprising cellulose acetate 398-10, NF triacetin, USP, polyethylene glycol 400, NF and acetone, NF to form Sodium Valproate XT tablets.

6. Laser drill an orifice of approximately 0.5 mm diameter on each side of the tablet using a laser drilling apparatus.

### EXAMPLE 2

Divalproex sodium sustained release tablets were prepared having the formulation in Table 2 below:

TABLE 2

| | P00365 | |
|---|---|---|
| Ingredient | mg/tablet | % w/w |
| Core Tablets | | |
| Sodium Valproate ^{(a)} | 576.00 | 58.01 |
| Sodium Hydroxide, NF | * | * |
| Lactose Anhydrous, USP | 191.88 | 19.32 |
| Microcrystalline Cellulose, NF | 49.65 | 5.00 |
| (Avicel PH-105 | | |
| Citric Acid Anhydrous, USP Fine | 140.00 | 14.10 |
| Granular | | |
| Cellulose Acetate Phthalate, NF | 11.15 | 1.12 |
| Polyethylene Glycol 400, NF | 2.23 | 0.22 |
| Colloidal Silicon Dioxide, NF | 12.41 | 1.25 |
| (Cab-O-Sil M5) | | |
| Magnesium Stearate, NF/FCC | 7.45 | 0.75 |
| Talc, USP (ALTALC 500V) | 2.23 | 0.22 |
| Purified Water, USP | ** | ** |
| Isopropyl Alcohol, USP | ** | ** |
| Acetone, NF | ** | ** |
| Sub-total: | 993.00 | 99.99 |
| Seal Coating | | |
| Opadry Clear (YS-1-7006) | 44.42 | 4.25 |
| Magnesium Stearate, NF/FCC | 7.84 | 0.75 |
| Ethanol-SDA 3A 190 Proof | ** | ** |
| Sub-total: | 1045.26 | 5.00^{(b)} |
| CA Coating | | |
| Cellulose Acetate 398-10, NF | 25.33 | 2.36 |
| Polyethylene Glycol 400, NF | 2.57 | 0.24 |
| Acetone, NF | ** | ** |
| Sub-total: | 1073.16 | 2.60^{(b)} |
| Total: | 1073.16 | |

* Less than 0.1% (w/w) is used to adjust the pH of solution to be more than 10.3
** Evaporated during processing
^{(a)}Core tablets contain neutralized divalproex sodium equivalent to 576.00 mg of sodium valproate, or 500 mg of valproic acid activity.
^{(b)}Total percentage for the specific step.

### EXAMPLE 3

Divalproex sodium sustained release tablets were prepared having the formulation in Table 3 below:

TABLE 3

| | P01145 | |
|---|---|---|
| Ingredient | mg/tablet | % w/w |
| Core Tablets | | |
| Sodium Valproate ^{(a)} | 576.00 | 58.01 |
| Sodium Hydroxide, NF | * | * |
| Lactose Anhydrous, USP | 191.88 | 19.32 |
| Microcrystalline Cellulose, NF | 49.65 | 5.00 |
| (Avicel PH-105 | | |
| Citric Acid Anhydrous, USP Fine | 140.00 | 14.10 |
| Granular | | |
| Cellulose Acetate Phthalate, NF | 11.15 | 1.12 |
| Polyethylene Glycol 400, NF | 2.23 | 0.22 |
| Colloidal Silicon Dioxide, NF | 12.41 | 1.25 |
| (Cab-O-Sil M5) | | |
| Magnesium Stearate, NF/FCC | 7.45 | 0.75 |
| Talc, USP (ALTALC 500V) | 2.23 | 0.22 |
| Purified Water, USP | ** | ** |
| Isopropyl Alcohol, USP | ** | ** |
| Acetone, NF | ** | ** |
| Sub-total: | 993.00 | 99.99 |
| Seal Coating | | |
| Opadry Clear (YS-1-7006) | 44.42 | 4.25 |
| Magnesium Stearate, NF/FCC | 7.84 | 0.75 |
| Ethanol-SDA 3A 190 Proof | ** | ** |
| Sub-total: | 1045.26 | 5.00^{(b)} |
| CA Coating | | |
| Cellulose Acetate 398-10, NF | 17.07 | 1.60 |
| Polyethylene Glycol 400, NF | 4.26 | 0.40 |
| Acetone, NF | ** | ** |
| Sub-total: | 1066.60 | 2.00^{(b)} |
| Total: | 1066.60 | |

* Less than 0.1% (w/w) is used to adjust the pH of solution to be more than 10.3
** Evaporated during processing
^{(a)}Core tablets contain neutralized divalproex sodium equivalent to 576.00 mg of sodium valproate, or 500 mg of valproic acid activity.
^{(b)}Total percentage for the specific step.

### EXAMPLE 4

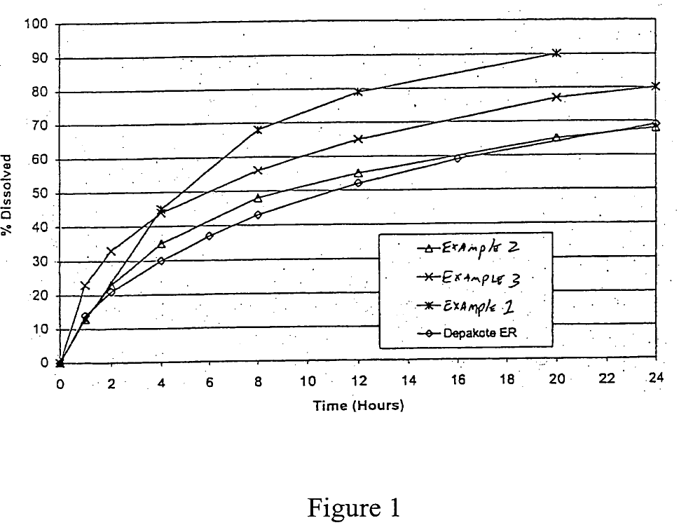

In-vitro dissolution was conducted on the formulations of Examples 2 and 3 (containing a solubility modulating agent) and the formulation of Example 1 (which does not contain a solubility modulating agent). As can be seen in the results in FIG. 1, the formulations of Examples 2 and 3 were faster than the formulation of Example 1.

### EXAMPLE 5

In vivo testing was done on tablets prepared in accordance with Example 1 and were compared to reference standard Depakote ER. The study was a 3 period cross-over study and the total number of subjects was 8. The mean results of the study are listed in table 5 below:

TABLE 5

| | | | | | | | 90% CI |
|---|---|---|---|---|---|---|---|
| | | Test Values | | Reference Values | | G-Mean | (non- |
| Condition | Parameters | Mean | CV (%) | Mean | CV (%) | Ratio | transformed) |
| Fasting | Cₘₐₓ | 19.83 | 25.98 | 22.17 | 34.60 | 0.912 | 65.2-114 |
| | AUC ₀₋ₜ | 517.64 | 33.35 | 613.39 | 40.37 | 0.882 | 65.4-103 |
| | Tₘₐₓ | 9.56 | 38.67 | 16.05 | 66.70 | 0.596 | |
| Fed | Cₘₐₓ | 22.97 | 34.77 | 30.36 | 27.57 | 0.739 | 57.4-93.9 |
| | AUC ₀₋ₜ | 649.74 | 25.54 | 782.57 | 31.63 | 0.840 | 72.8-93.2 |
| | Tₘₐₓ | 12.98 | 40.29 | 12.44 | 32.41 | 1.043 | |

### EXAMPLE 6

In vivo testing was done on tablets prepared in accordance with Example 1 and were compared to reference standard Depakote ER. The study was a 3 period cross-over study and the total number of subjects was 8. The mean results of the study are listed in table 6 below:

TABLE 6

| | | | | | | | 90% CI |
|---|---|---|---|---|---|---|---|
| | | Test Values | | Reference Values | | G-Mean | (non- |
| Condition | Parameters | Mean | CV (%) | Mean | CV (%) | Ratio | transformed) |
| Fasting | Cₘₐₓ | 32.15 | 24.33 | 22.17 | 34.60 | 1.522 | 121-169 |
| | AUC ₀₋ₜ | 730.28 | 21.20 | 613.39 | 40.37 | 1.300 | 100-138 |
| | Tₘₐₓ | 6.59 | 31.43 | 16.05 | 66.70 | 0.411 | |
| Fed | Cₘₐₓ | 43.63 | 24.40 | 30.36 | 27.57 | 1.465 | 125-162 |
| | AUC ₀₋ₜ | 909.68 | 19.77 | 782.57 | 31.63 | 1.206 | 106-126 |
| | Tₘₐₓ | 5.42 | 16.83 | 12.44 | 32.41 | 0.436 | |

The examples provided above are not meant to be exclusive. Many other variations of the present invention would be obvious to those skilled in the art, and are contemplated to be within the scope of the appended claims. For example, it will be recognized by those skilled in the art that a wide variety of pharmaceutically acceptable excipients may be utilized for their intended purpose in the process for preparing divalproex sodium sustained release oral dosage forms as described herein.

1. A sustained release oral dosage form comprising:
a therapeutically effective dose of neutralized divalproex sodium;
a solubility modulating agent; and
a pharmaceutically acceptable carrier;
said sustained release oral dosage form providing a Tₘₐₓ at from about 4 to about 20 hours after oral administration of the dosage form to a human.

2. The sustained release oral dosage form of claim 1 wherein said therapeutically effective dose of neutralized divalproex sodium, said solubility modulating agent, and said pharmaceutically acceptable carrier is compressed into a tablet core.

3. The sustained release oral dosage form of claim 2, wherein said tablet core is coated with a membrane coating.

4. The sustained release oral dosage form of claim 3, wherein said membrane coating has a passageway disposed therein for the passage of the neutralized divalproex sodium upon exposure to an environmental fluid.

5. The sustained release oral dosage form of claim 1, wherein said solubility modulating agent in overcoated with a water-insoluble coat.

6. The sustained release oral dosage form of claim 5, wherein said water-insoluble coat is selected from the group consisting of Eudragit LD, shellac, cellulose acetate butyrate, hydroxypropyl methylcellulose phthalate, cellulose acetyl phthalate, cellulose triacetyl phthalate, sodium cellulose acetate phthalate, cellulose ester phthalate, cellulose ether phthalate, methylcellulose phthalate, cellulose ester-ether phthalate, hydroxy propyl cellulose phthalate, alkali salts of cellulose acetate phthalate, alkaline earth salts of cellulose acetate phthalate, calcium salt of cellulose acetate phthalate, ammonium salt of hydroxypropyl methylcellulose phthalate, cellulose acetate hexahydrophthalate, hydroxypropyl methylcellulose hexahydrophthalate, polyvinyl acetate phthalate, and mixtures thereof.

7. The sustained release oral dosage form of claim 3, wherein said membrane coating comprise a polymer selected from the group consisting of cellulose acetate, cellulose acetate acetoacetate, cellulose acetate chloroacetate, cellulose acetate furoate, dimethoxyethyl cellulose acetate, cellulose acetate carboxymethoxypropionate, cellulose acetate benzoate, cellulose butyrate naphthylate, cellulose acetate benzoate, methylcellulose acetate, methylcyanoethyl cellulose, cellulose acetate methoxyacetate, cellulose acetate ethoxyacetate, cellulose acetate dimethylsulfamate, ethylcellulose, ethylcellulose dimethylsulfamate, cellulose acetate p-toluene sulfonate, cellulose acetate methylsulfonate, cellulose acetate dipropylsulfamate, cellulose acetate butylsulfonate, cellulose acetate laurate, cellulose stearate, cellulose acetate methylcarbamate, agar acetate, amylose triacetate, beta glucan acetate, beta glucan triacetate, acetaldehyde dimethyl acetate, cellulose acetate ethyl carbamate, cellulose acetate phthalate, cellulose acetate dimethyl aminoacetate, cellulose acetate ethyl carbonate, poly (vinyl methyl) ether copolymers, cellulose acetate with acetylated hydroxyethyl cellulose, hydroxylated ethylenevinyl acetate, poly(ortho ester)s, polyacetals, semipermeable polyglycolic, polylactic acid, film forming materials with a water sorption of one to fifty percent by weight at ambient temperatures with a presently preferred water sorption of less than thirty percent, acylated polysaccharides, acylated starches, aromatic nitrogen containing polymeric materials that exhibit permeability to aqueous fluids, membranes made from polymeric epoxides, copolymers of alkylene oxides and alkyl glycidyl ethers, polyurethanes, polyacrylate and polymethacrylate polymers and mixtures thereof.

8. The sustained release oral dosage form of claim 3, wherein said membrane coating comprises cellulose acetate.

9. The sustained release oral dosage form of claim 3, wherein said membrane coating comprises a plasticizer.

10. The sustained release oral dosage form of claim 9, wherein said plasticizer is selected from the group consisting of phthalates, phosphates, citrates, adipates, tartrates, sebacates, succinates, glycolates, glycerolates, benzoates, myristates, polyethylene glycols, polypropylene glycols, halogenated phenyls, triacetin, acetylated monoglyceride, grape seed oil, olive oil, sesame oil, acetyltributylcitrate, acetyltriethylcitrate, glycerin sorbitol, diethyloxalate, diethylmalate, diethylfumarate, dibutylsuccinate, diethylmalonate, dioctylphthalate, dibutylsebacate, triethylcitrate, tributylcitrate, glyceroltributyrate, and mixtures thereof.

11. The sustained release oral dosage form of claim 1, wherein said solubility modulating agent is selected from the group consisting organic carboxylic acids; acid anhydrides; acid salts; carbonate sources; and mixtures thereof.

12. The sustained release oral dosage form of claim 1, wherein said solubility modulating agent is citric acid.

13. A process for preparing an oral sustained release dosage form comprising:
(a) preparing a neutralized divalproex sodium solution by combining divalproex sodium, having a sodium valproate moiety and a valproic acid moiety, with a base and an aqueous solvent, the base being added in sufficient amount to ensure neutralization of the valproic acid moiety of the divalproex sodium,
(b) combining the neutralized divalproex sodium solution with a solubility modulating agent; and
(c) forming a sustained release dosage form.

14. The process of claim 13, further comprising coating the solubility modulating agent with a water insoluble coating.

15. The process of claim 13, wherein said base is selected from the group consisting of sodium carbonate, sodium bicarbonate, sodium phosphate dibasic, sodium phosphate tribasic, sodium citrate, magnesium hydroxide, magnesium carbonate, calcium carbonate, calcium phosphate, sodium hydroxide and mixtures thereof.

16. The process of claim 13, wherein said base is sodium hydroxide.

17. The process of claim 13, further comprising granulating said neutralized divalproex solution with a pharmaceutically acceptable carrier prior to combining with said solubility modulating agent.

18. The process of claim 17, wherein said granulating is spray granulating.

19. The process of claim 13, wherein said solubility modulating agent is coated with a water insoluble coating prior to mixing with the neutralized divalproex sodium.

20. The process of claim 13, further comprising adding one or more pharmaceutically necessary tableting excipients to the mixture.

21. The process of claim 20, further comprising compressing the mixture into a tablet or tablet core.

22. The process of claim 21, further comprising applying a membrane coating to the tablet core.

23. The process of claim 21, further comprising forming a passageway in the coating of the tablet.

24. The oral dosage form of claim 21.

25. The process of claim 13, wherein said solubility modulating agent is selected from the group consisting organic carboxylic acids; acid anhydrides; acid salts; carbonate sources; and mixtures thereof.

26. The process of claim 13, wherein said solubility modulating agent is citric acid.

27. A method of treating and/or preventing complex partial seizures, mania associated with bipolar disorders and migraine headaches comprising administering a dosage form of claim 1 to a human patient in need of treatment with said dosage form.
